(19) United States
(12) Patent Application Publication
(10) Pub. No.: US 2016/0121136 A1
(43) Pub. Date: May 5, 2016

(54) PHOTOTHERAPY TEXTILE WRAPPER

(71) Applicant: THE HONG KONG RESEARCH INSTITUTE OF TEXTILES AND APPAREL LIMITED, Hong Kong (HK)

(72) Inventors: Xiaoming TAO, Hong Kong (HK); Wei ZHENG, Hong Kong (HK); Zhifeng ZHANG, Hong Kong (HK); Hoiching LAU, Hong Kong (HK); Laiwa LEE, Hong Kong (HK)

(21) Appl. No.: 14/534,153

(22) Filed: Nov. 5, 2014

(51) Int. Cl. A61N 5/06

(52) U.S. Cl. CPC A61N 5/0621; A61N 2005/063; A61N 2005/0663; A61N 2005/0651; A61N 2005/067

(57) ABSTRACT

A therapeutic textile wrapper configured to project therapeutic light to a person is provided. The wrapper has a back layer on which there are one or more luminescent fabric layers configured to irradiate the therapeutic light. An individual luminescent fabric layer further overlies a reflective layer positioned on the back layer. Preferably, the wrapper includes a cover fabric layer on the one or more luminescent fabric layers. The individual luminescent fabric layer comprises one or more yarns and one or more optical fibers (OFs) woven together to form a main body of the individual luminescent fabric layer. The portion of the individual OF residing inside the main body has a lateral surface for allowing light rays traveling inside the individual OF to at least partially leak out therefrom through the lateral surface. The individual OF is configured to be optically connectable to a light source for receiving the therapeutic light.

### FIELD OF THE INVENTION

The present invention relates to a phototherapy textile wrapper for projecting therapeutic light to a person.

### BACKGROUND

Neonatal hyperbilirubinemia (jaundice) is distinguished by an increased level of unconjugated bilirubin in a neonate's blood. Bilirubin is a toxic molecule, which is produced in recycling iron from breaking down a heme group in hemoglobin of worn-out red blood cells. The livers of some newborns are not mature enough to conjugate bilirubin, so that this toxic substance cannot be excreted and cleared from the newborns' bodies. Accumulation of bilirubin results in hyperbilirubinemia, leading to yellowing of skin and other tissues. Severe jaundice may cause brain damage and even death. It has been reported that in every year, about 60 percent of infants and 80 percent of premature infants in the US become clinically jaundiced during the first week after birth. Currently, it is believed that phototherapy is an effective method for treating neonatal hyperbilirubinemia with no significant side effects.

Phototherapy turns unconjugated bilirubin into water-soluble compounds, which are easier to excrete via kidneys than unconjugated bilirubin does. There are three critical reactions in the transformation of bilirubin, namely, photo-oxidation, configurational isomerization and structural isomerization. During structural isomerization, bilirubin is transformed to a water-soluble compound, lumirubin, which is non-toxic and can be easily excreted from an infant's body.

The optimal wavelength for phototherapy ranges from 450 nm to 460 nm. Light having such optimal wavelength has a blue color in the visible spectrum. Apart from the wavelength, the light's intensity (which depends on the distance between a light source and the infant), the length of exposure time and an area of exposure of the light to the infant's body also affect the therapeutic effect of phototherapy.

Commonly-found commercial phototherapy systems can be classified into two types, viz., a box type and a blanket type. The blue light box system is deployable in hospitals. Fluorescent lamps or light emitting diode (LED) lamps are used as light sources to emit light of desirable wavelengths for the treatment. One or more such light sources are usually suspended over an affected infant, which is put into an incubator or a bassinet. This therapeutic strategy, which employs strong light having a high intensity, is common in hospitals. Sometimes an optical fiber pad, exemplarily formed by slipping optical fibers into a transparent plastic bag during usage, is placed under the infant to provide a better coverage of the light. However, such light-irradiation system has significant drawbacks. First, the treatment based on this system is rather expensive. Also, the bulkiness and a high price of the equipment make it difficult to use such therapeutic strategy for home treatment. During the treatment, the infant is separated from its parents and is placed alone inside the box with its eyes masked, causing discomfort and tension to both the parents and the infant. Furthermore, some designs of the light box provide only a unidirectional light source because, for example, fluorescent lamps can only be suspended above the infant, reducing exposure of available skin area to the light. A fluorescent lamp, which is a commonly-used light source in the box system, produces a significant amount of heat and can cause adverse side effects such as water-loss and hyperthermia. In addition, the fluorescent lamp can lead to potentially harmful effects to the infant on biological rhythms. It is furthermore noted that the use of the transparent plastic bag for the optical fiber pad reduces ventilation, causing discomfort to the infant.

Treatments using blanket devices have become increasingly popular. Each of such devices usually uses a flexible optical fiber pad comprising a LED light source, which provides a sufficiently-high light intensity in the desired wavelength. The devices are comparably cheaper and portable designs of these devices make home treatment possible. The blanket design can avoid separation of the infant and its parents. Close contact between the infant and the light source avoids wastage of the light energy. At present, there are several commercial phototherapy blankets available, for example, Biliblanket Phototherapy system, Bilisoft Phototherapy system and the Bilirubin Blanket.

Nevertheless, currently available products have significant limitations. Although designs such as Biliblanket Phototherapy system and Bilisoft Phototherapy system provide a sufficient irradiation area, they can only be placed under the infant, thereby limiting the exposed skin area of the infant for the treatment. Bilirubin Blanket uses blue LEDs built in a woven fabric blanket so as to allow comfortable wrap-up and a better coverage of the light source to be achieved. However, the design of built-in LEDs creates difficulties in cleaning and sterilization.

Therefore, there is a need in the art for a phototherapy device providing a sufficiently-high light intensity with a maximized coverage of skin for medical treatment. Comfort, air permeability and sterilizing problems are also needed to be considered.

### SUMMARY OF THE INVENTION

The present invention provides a phototherapy textile wrapper configured to project therapeutic light to a person. The wrapper comprises a back layer, and one or more luminescent fabric layers positioned on a same side of the back layer and configured to irradiate the therapeutic light. An individual luminescent fabric layer overlies a reflective layer that is used for substantially reflecting the therapeutic light received thereon. The reflective layer is arranged to be sandwiched between the individual luminescent fabric layer and the back layer. The individual luminescent fabric layer comprises one or more yarns and one or more optical fibers (OFs). The one or more yarns and the one or more OFs are woven together to form a main body of the individual luminescent fabric layer. In one embodiment, the one or more yarns may include a nylon/cotton yarn. An individual OF extends outside the main body. The portion of the individual OF residing inside the main body has a lateral surface configured to allow light rays traveling inside the individual OF to at least partially leak out therefrom through the lateral surface. The portion of the individual OF extending outside the main body is configured to be optically connectable to a light source for receiving the therapeutic light generated from the light source such that positioning the light source away from the wrapper is allowable. Thereby, discomfort caused to the person by the light source's generated heat is avoidable.

Preferably, the wrapper further comprises a cover fabric layer overlying the one or more luminescent fabric layers. The cover fabric layer is configured to at least partially transmit the therapeutic light that passes therethrough.

In one embodiment, the individual OF is a polymer optical fiber (POF). The POF may have a diameter in a range of 50 μm to 2,000 μm.

The main body of the individual luminescent fabric layer is preferably pre-processed by a hot press method to increase leakage of the light rays through the lateral surface. The hot press method comprises pressing the main body with a pressure from 2 bars to 8 bars at a temperature from 50° C. to 120° C. for a duration from 5 s to 60 s. The leakage may also be increased by making notches on the lateral surface by laser ablation, electric beam irradiation, chemical etch, mechanical pressing, or cutting.

The individual OF may be configured to at least transmit a light beam having a wavelength from 450 nm to 460 nm, thereby configuring the wrapper to be used for treating neonatal jaundice for the person.

It is desirable and advantageous that the individual luminescent fabric layer is configured to be detachable from the back layer, thereby allowing the one or more luminescent fabric layers and the rest of the wrapper to be separately cleaned or sterilized.

Preferably, the back layer is configured to substantially block the therapeutic light from passing through. As a result, a protective mask for covering the person's eyes can be eliminated during receiving the therapeutic light. In addition, discomfort caused by the therapeutic light to the medical staff can be avoided.

Other aspects of the present invention are disclosed as illustrated by the embodiments hereinafter.

### BRIEF DESCRIPTION OF THE DRAWINGS

FIG. 1 depicts a structure of a phototherapy textile wrapper as disclosed herein in accordance with an exemplary embodiment of the present invention, where the wrapper has one or more luminescent fabric layers configured to irradiate therapeutic light to a person, such as a neonatal infant, when the person is wrapped with the wrapper.

FIG. 2 depicts, in accordance with the exemplary embodiment of the present invention, a layer-by-layer structure of an individual luminescent fabric layer, where a main body of the individual luminescent fabric layer is formed by weaving one or more yarns and one or more optical fibers together, and the individual optical fiber extends outside the main body to receive the therapeutic light from a light source.

FIG. 3 is a microscope image that depicts a cross section of one realization of the individual luminescent fabric layer, illustrating that the one or more yarns and the one or more optical fibers are woven together to form the main body of the individual luminescent fabric layer.

FIG. 4 is an enlarged diagram of a polymer optical fiber with the lateral surface thereof treated in order to increase the percentage of therapeutic light traveling inside the polymer optical fiber to leak out therefrom.

### DETAILED DESCRIPTION

An aspect of the present invention is to provide a phototherapy textile wrapper configured to project therapeutic light to a person. It is an aim that the wrapper as provided is configured to be flexible, air-permeable and sterilizable. An exemplary application of the wrapper is that the person receives the therapeutic light when the person is wrapped with the wrapper. The wrapper is usable in a therapeutic process of treating neonatal jaundice of an infant patient. Nevertheless, it does not imply that the wrapper is limited only for treating jaundice. Furthermore, although wrapping the whole body of the infant patient with the wrapper is preferable for treating neonatal jaundice, the wrapper as disclosed herein is not limited only to be configured to wrap the whole body of the person who receives the therapeutic light. The wrapper may be configured to wrap only a part of the body, such as a torso or an arm of the person, when the person receives the therapeutic light.

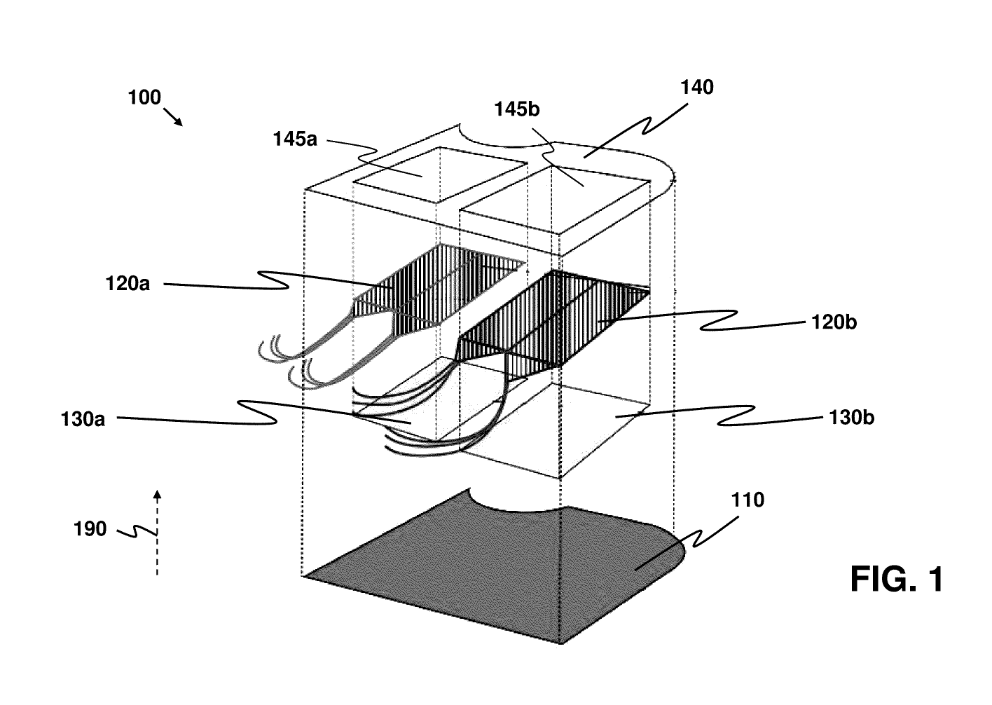

The phototherapy textile wrapper is exemplarily illustrated with an aid of FIG. 1, which depicts a layer-by-layer structure of a phototherapy textile wrapper 100 according to an exemplary embodiment of the present invention. The wrapper 100 comprises plural layers and is formed by integrating the plural layers together. For the sake of convenience and clarity in illustration, a reference vertical direction 190 is used herein as a reference in illustrating and defining positions of the plural layers.

The wrapper 100 comprises a back layer 110, and one or more luminescent fabric layers 120a, 120b positioned on a same side of the back layer 110 and configured to irradiate the therapeutic light. Although FIG. 1 shows two luminescent fabric layers, the present invention is not limited to having only two such layers. In addition, an individual luminescent fabric layer 120a (120b) overlies a reflective layer 130a (130b) for substantially reflecting the therapeutic light received on the reflective layer 130a (130b). The reflective layer 130a (130b) is sandwiched between the individual luminescent fabric layer 120a (120b) and the back layer 110. Despite FIG. 1 depicts that physically separate reflective layers 130a, 130b are used for different individual luminescent fabric layers 120a, 120b, it is possible that the reflective layers 130a, 130b may be substituted by one sufficiently-long contiguous reflective layer positioned below the two individual luminescent fabric layers 120a, 120b.

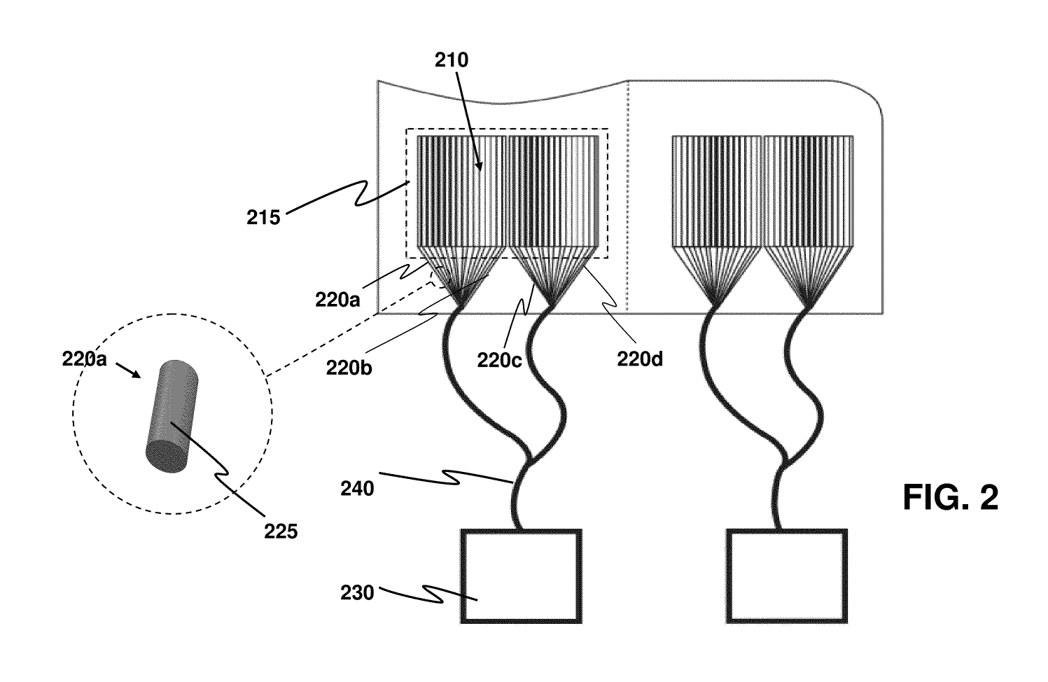

An exemplarily embodiment of the individual luminescent fabric layer 120a (120b) is detailed as follows with an aid of FIG. 2. An individual luminescent fabric layer 210 has a main body 215. As used herein, a main body of a luminescent fabric layer means a portion of the luminescent fabric layer configured to irradiate a substantial portion of therapeutic light. The individual luminescent fabric layer 210 comprises one or more yarns and one or more optical fibers (OFs), where the one or more yarns and the one or more OFs are woven together to form the main body 215. It results in the main body 215 that is flexible and soft while providing air permeability so as to provide more comfort to the person as compared to existing wrapper designs formed by plastic sheets, encouraging the person to keep close contact to the wrapper 100 to thereby receive more therapeutic light for promoting treatment effects. In one option, the one or more yarns include a nylon/cotton yarn. In a further option, the one or more yarns may be made of nylon/cotton.

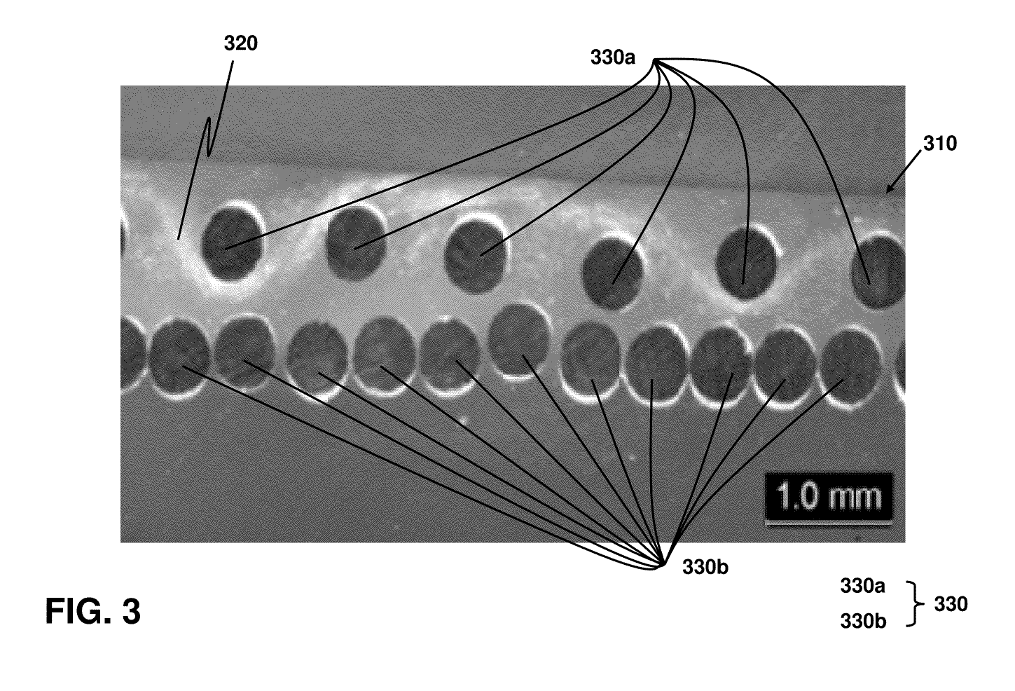

As one illustrative example, FIG. 3 is a microscope image depicting one practical realization of an individual luminescent fabric layer, and provides a cross-sectional view of a main body 310 thereof. The main body 310 has first plural OFs 330a in one layer and second plural OFs 330b in another layer, providing a double-layer structure for forming the main body 310. Although the double-layer structure is used here for illustration, it is possible that the main body 310 is realizable with just one single layer or more than two layers. As shown in FIG. 3, OFs 330 in both of the layers (incorporating both the first plural OFs 330a and the second plural OFs 330b) are woven with yarns 320. The yarns 320 and the OFs 330 are co-arranged to be approximately orthogonal to each other in order that the yarns 320 and the OFs 330 are woven and integrated together to form the main body 310.

In FIG. 2, the one or more OFs as mentioned above are indicated as individual OFs 220a, 220b, 220c and 220d as examples for illustration. An individual OF, selected from the one or more OFs 220a-220d, extends outside the main body 215. In the description that follows, the individual OF is referenced as 220a for simplicity. The portion of the individual OF 220a residing inside the main body 215 has a lateral surface 225 configured to allow light rays traveling inside the individual OF 220a to at least partially leak out therefrom through the lateral surface 225. The portion of the individual OF 220a extending outside the main body 215 is configured to be optically connectable to a light source 230 for receiving the therapeutic light generated from the light source 230 such that positioning the light source 230 away from the wrapper 100 is allowable. It is an important advantage of the wrapper 100 in that discomfort caused to the person by the generated heat of the light source 230 is avoidable. In one practical implementation, the light source 230 provides the therapeutic light to the individual OF 220a via an optical cable 240. As an example, the light source 230 may be a LED or a laser-generating device.

If the wrapper 100 is used for treating neonatal jaundice for the person, the individual OF 220a is configured to at least transmit a light beam having a wavelength from 450 nm to 460 nm. For treating another disease, the individual OF 220a is configured to at least transmit therapeutic light of a range of wavelength corresponding to treating this disease. In one option, the individual OF 220a may be configured to transmit light of wavelength from 500 nm to 1200 nm so that the wrapper 100 has potential applications in skin pigment removal or other phototherapy.

Preferably, the individual OF 220a is a polymer optical fiber (POF). If it is desired to use the therapeutic light of wavelength from 450 nm to 460 nm used for phototherapy of neonatal jaundice, the POF may be selected to have a diameter in a range of 50 μm to 2,000 μm.

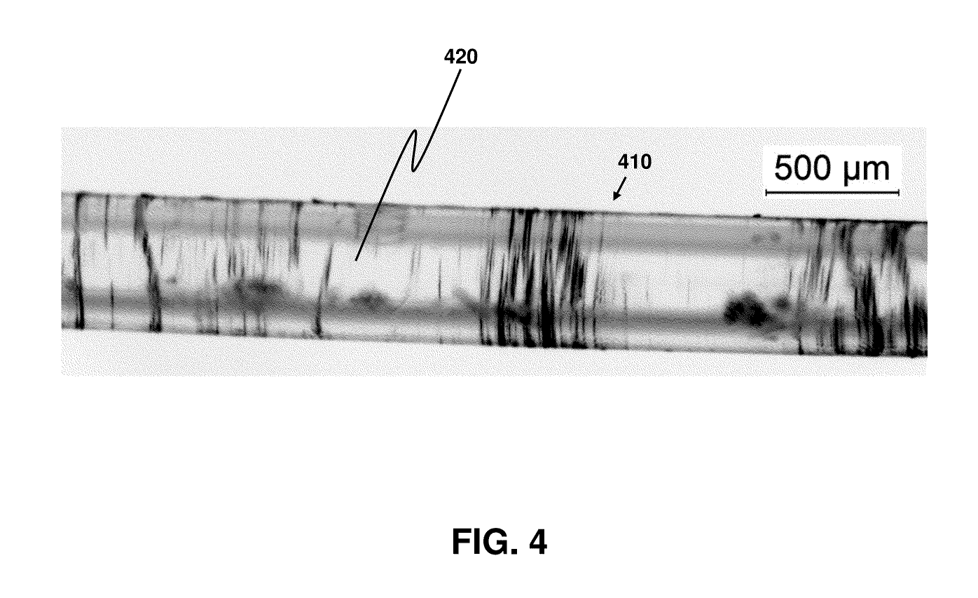

As mentioned above, the lateral surface 225 of the individual OF 220a that resides in the main body 215 is configured to at least partially leak out the light rays traveling inside the individual OF 220a. To increase the irradiation level of the light rays leaking out from the lateral surface 225, one option is to pre-process the main body 215 by a hot press method. By the hot press method, the main body 215 is pressed with a pressure from 2 bars to 8 bars at a temperature from 50° C. to 120° C. for a duration from 5 s to 60 s. FIG. 4 is an enlarged diagram providing an example of a POF 410 treated with the hot press method. It is shown that the POF 410 has a lateral surface 420 that is roughened, thereby causing more light to escape from inside the POF 410.

Apart from the hot press method, said irradiation level can also be increased by making notches on the lateral surface 225 of the individual OF 220a residing in the main body 215 by laser ablation, electric beam irradiation, chemical etch, mechanical pressing, or cutting.

Refer to FIG. 1 again. To provide flexibility and air permeability to the wrapper 100 so as to provide comfort to the person and to enable the wrapper 100 to be foldable for wrapping the person, preferably the back layer 110 is formed substantially by one or more textile materials such as a fabric. The back layer 110 may be realized in a form of a blanket. Moreover, the reflective layer 130a (130b) may be made of fabric having a substantially reflective surface.

It is preferable that the wrapper 100 further comprises a cover fabric layer 140 overlying the individual luminescent fabric layer 120a (120b). The cover fabric layer 140 is configured to at least partially transmit the therapeutic light that passes therethrough. In one practical realization, the cover fabric layer 140 comprises openings 145a, 145b positioned directly over the individual luminescent fabric layers 120a, 120b for allowing the therapeutic light produced by these luminescent fabric layers 120a, 120b and/or reflected by the reflective layers 130a, 130b to pass through the cover fabric layer 140 without obstruction.

The positional arrangement of the cover fabric layer 140, the one or more luminescent fabric layers 120a, 120b, the reflective layers 130a, 130b and the back layer 110 indicates that the person may receive the therapeutic light when the person's skin is intimately contacting the cover fabric layer 140 and the one or more luminescent fabric layers 120a, 120b. Comfort to the person is obtained as the cover fabric layer 140 and the one or more luminescent fabric layers 120a, 120b are advantageously formed by including textile fabrics.

Preferably, the back layer 110 is configured to substantially block the therapeutic light from passing through. Thereby, a protective mask for covering the person's eyes can be eliminated during receiving the therapeutic light. Discomfort caused by the therapeutic light to the medical staff can also be avoided. The presence of the cover fabric layer 140 in the wrapper 100 has an advantage that a material different from the cover fabric layer 140 may be used to make the back layer 110 because the back layer 110 does not need to directly, intimately contact the person. The material for making the back layer 110 may be optimized for effectiveness in blocking the therapeutic light.

Advantageously and desirably, the individual luminescent fabric layer 120a (120b) is configured to be detachable from the back layer, thereby allowing the one or more luminescent fabric layers 120a, 120b and the rest of the wrapper 100 to be separately cleaned or sterilized. Greater convenience in cleaning and sterilization over existing phototherapy blankets having built-in LEDs integrated into the blankets is another advantage of the wrapper 100.

A phototherapy device is realizable by including a light source for generating therapeutic light, and the phototherapy textile wrapper as set forth in any of the embodiments disclosed above, wherein the wrapper is further configured to optically couple to the light source for receiving the therapeutic light generated therefrom. If the phototherapy device is for treating neonatal jaundice, the light source is further configured such that a substantial portion of the therapeutic light that is generated has a wavelength from 450 nm to 460 nm.

The present invention may be embodied in other specific forms without departing from the spirit or essential characteristics thereof. The present embodiment is therefore to be considered in all respects as illustrative and not restrictive. The scope of the invention is indicated by the appended claims rather than by the foregoing description, and all changes that come within the meaning and range of equivalency of the claims are therefore intended to be embraced therein.

What is claimed is:

1. A phototherapy textile wrapper configured to project therapeutic light to a person, comprising:
a back layer; and
one or more luminescent fabric layers positioned on a same side of the back layer and configured to irradiate the therapeutic light, an individual luminescent fabric layer overlying a reflective layer used for substantially reflecting the therapeutic light received thereon where the reflective layer is arranged to be sandwiched between the individual luminescent fabric layer and the back layer;
wherein:
the individual luminescent fabric layer comprises one or more yarns and one or more optical fibers (OFs), the one or more yarns and the one or more OFs being woven together to form a main body of the individual luminescent fabric layer, an individual OF extending outside the main body;
the portion of the individual OF residing inside the main body has a lateral surface configured to allow light rays traveling inside the individual OF to at least partially leak out therefrom through the lateral surface; and
the portion of the individual OF extending outside the main body is configured to be optically connectable to a light source for receiving the therapeutic light generated from the light source such that positioning the light source away from the wrapper is allowable, thereby allowing discomfort caused to the person by the light source's generated heat to be avoidable.

2. The wrapper of claim 1, wherein the individual OF is a polymer optical fiber (POF).

3. The wrapper of claim 2, wherein the POF has a diameter in a range of 50 μm to 2,000 μm.

4. The wrapper of claim 2, wherein the lateral surface is configured to allow light rays traveling inside the individual OF to at least partially leak out therefrom through the lateral surface by pre-processing the main body of the individual luminescent fabric layer by a hot press method, the hot pressing method comprising: pressing the main body with a pressure from 2 bars to 8 bars at a temperature from 50° C. to 120° C. for a duration from 5 s to 60 s.

5. The wrapper of claim 2, wherein the lateral surface is configured to allow light rays traveling inside the individual OF to at least partially leak out therefrom through the lateral surface by making notches on the lateral surface by laser ablation, electric beam irradiation, chemical etch, mechanical pressing, or cutting.

6. The wrapper of claim 1, wherein the individual OF is configured to at least transmit a light beam having a wavelength from 450 nm to 460 nm, thereby configuring the wrapper to be used for treating neonatal jaundice for the person.

7. The wrapper of claim 1, wherein the one or more yarns include a nylon/cotton yarn.

8. The wrapper of claim 1, wherein the individual luminescent fabric layer is configured to be detachable from the back layer, thereby allowing the one or more luminescent fabric layers and the rest of the wrapper to be separately cleaned or sterilized.

9. The wrapper of claim 1, wherein the back layer is configured to substantially block the therapeutic light from passing through.

10. The wrapper of claim 1, wherein the back layer is a fabric.

11. The wrapper of claim 1, wherein the back layer is a blanket.

12. The wrapper of claim 1, further comprising:
a cover fabric layer overlying the one or more luminescent fabric layers, the cover fabric layer being configured to at least partially transmit the therapeutic light that passes therethrough.

13. The wrapper of claim 12, wherein the cover fabric layer has one or more openings such that the therapeutic light is allowed to pass through the cover fabric layer via passing through the one or more openings.

14. The wrapper of claim 1, wherein the portion of the individual OF extending outside the main body is further configured to be optically connectable to the light source via an optical cable.

15. A phototherapy device comprising:
a light source for generating therapeutic light; and
the wrapper of claim 1 wherein the wrapper is further configured to optically couple to the light source for receiving the therapeutic light generated therefrom.

16. The phototherapy device of claim 15, wherein the therapeutic light has a wavelength from 450 nm to 460 nm.

17. The phototherapy device of claim 15, wherein the light source is a light emitting diode (LED) or a laser-generating device.

18. A phototherapy device comprising:
a light source for generating therapeutic light; and
the wrapper of claim 2 wherein the wrapper is further configured to optically couple to the light source for receiving the therapeutic light generated therefrom.

19. A phototherapy device comprising:
a light source for generating therapeutic light; and
the wrapper of claim 12 wherein the wrapper is further configured to optically couple to the light source for receiving the therapeutic light generated therefrom.

20. A phototherapy device comprising:
a light source for generating therapeutic light, a substantial portion of which has a wavelength from 450 nm to 460 nm; and
the wrapper of claim 6 wherein the wrapper is further configured to optically couple to the light source for receiving the therapeutic light generated therefrom.
